(19) United States
(12) Patent Application Publication
(10) Pub. No.: US 2025/0095248 A1
(43) Pub. Date: Mar. 20, 2025

(54) METHOD AND SYSTEM FOR ALTERNATELY DISPLAYING GRAPHICAL REPRESENTATIONS

(71) Applicant: Siemens Healthineers AG, Forchheim (DE)

(72) Inventor: Philipp Bernhardt, Forchheim (DE)

(21) Appl. No.: 18/726,704

(22) PCT Filed: Dec. 13, 2022
(86) PCT No.: PCT/EP2022/085536

§ 371 Date: Jul. 3, 2024

(30) Foreign Application Priority Data
Jan. 19, 2022 (DE) 10 2022 200 571.6

(51) Int. Cl. G06T 11/60; G06T 7/00

(52) U.S. Cl. CPC G06T 11/60; G06T 7/0014; G06T 2207/20092; G06T 2207/30004; G06T 2210/41

(57) ABSTRACT

A method for alternately displaying graphical representations includes: a) providing a first image that includes a presentation of an anatomical structure of an object of interest; b) providing a second image that reproduces a spatial and/or time-related change in the object of interest; c) displaying a graphical representation of the first or second image on a display surface of a display unit; and d) displaying a graphical representation of the respective other image on the display surface of the display unit, wherein acts c) and d) are carried out one after the other and acts b) to d) are carried out repeatedly.

The present patent document is a § 371 nationalization of PCT Application Serial No. PCT/EP2022/085536, filed Dec. 13, 2022, designating the United States, and this patent document also claims the benefit of German Patent Application No. 10 2022 200 571.6, filed Jan. 19, 2022, which are incorporated by reference in their entireties.

### TECHNICAL FIELD

The present disclosure relates to a method for alternately displaying graphical representations, to a system, and to a computer program product.

### BACKGROUND

Time-resolved imaging methods are frequently used to detect changes in an object under examination, (e.g., a human and/or animal patient). The change to be detected may include movement of a contrast agent and/or of a medical object in the object under examination, in particular in a region of interest of the object under examination. To improve the mapping of the change in the object under examination, a differential imaging method, in particular an X-ray-based technique such as digital subtraction angiography (DSA), may be used. In DSA, a mask image may be subtracted from one or more fill images and a difference image is provided, wherein the mask image maps the object under examination without the contrast agent and/or medical object, and the at least one fill image shows the object under examination with the contrast agent and/or medical object present. To map the temporal dynamics of the change in the object under examination, a plurality of fill images may be acquired in chronological order and combined with the mask image to form a time-resolved difference image. In this way, spatially and/or temporally unchanging areas of the object under examination that are shown in the mask image and the at least one fill image may be removed from the difference image.

In medical imaging, in particular X-ray imaging, the roadmapping technique may be used. Similarly to digital subtraction angiography, a vascular image is first created. This vascular image is then superimposed on a subtracted fluoroscopic image, e.g., inverted. With this technique, a user, (e.g., a medical staffer), may use the vascular image for orientation when inserting medical objects, (e.g., guidewires), and thus better determine their path. The superimposition of two subtracted grayscale images may result in structures canceling each other out and thus no longer being visible. In addition, the noise increases additively, and the information may no longer be assigned to the individual subtraction images.

These omissions may be reduced, for example, by using color images and/or special mixing processes. Color images appear somewhat less contrastive to the eye than grayscale images and an interpretative intervention in the image is inevitably required, as well as an enhancement or weakening of given contrasts, which may result in misinterpretations if the user, in particular the medical staffer, draws conclusions from a contrast intensity.

### SUMMARY AND DESCRIPTION

The object of the disclosure is therefore to provide improved visual monitoring of spatial and/or time-related changes in an object under examination.

The scope of the present disclosure is defined solely by the appended claims and is not affected to any degree by the statements within this summary. The present embodiments may obviate one or more of the drawbacks or limitations in the related art.

In a first aspect, the disclosure relates to a method for alternately displaying graphical representations. In act a), a first image is provided which has a mapping of an anatomical structure of an object under examination. In addition, in act b), a second image is provided which maps a spatial and/or time-related change in the object under examination. In act c), a graphical representation of the first or the second image is displayed on a display surface of a display unit. In act d), a graphical representation of the respective other image is displayed on the display surface of the display unit. In addition, acts c) and d) are carried out in chronological succession, and acts b) to d) are carried out repeatedly.

Providing the first and/or the second image may include receiving and/or acquiring the respective image. In particular, receiving the first and/or second image may include recording and/or reading from a computer-readable data memory and/or receiving from a data storage unit, for example, a database. For example, the first and/or second image may be provided by a provision unit of a medical imaging device for acquiring the first and/or second image. Alternatively, or in addition, the first and second images may be acquired by one or more medical imaging devices. The at least one medical imaging device for acquiring the first and second images may be a magnetic resonance imaging (MRI) installation, a computed tomography (CT) installation, a medical X-ray device, a positron emission tomography (PET) installation, an ultrasound device, or combination thereof.

The first image may map the anatomical structure of the object under examination in a two-dimensional (2D) and/or three-dimensional (3D) spatially resolved manner. The first image may also map the anatomical structure of the object under examination in a time-resolved manner. In particular, the first image may include a contrasted and/or segmented mapping of the object under examination, in particular of the anatomical structure. The first image may advantageously include a plurality of first picture elements, in particular pixels and/or voxels. The object under examination may be a human and/or animal patient. The anatomical structure may be an organ, (e.g., a hollow organ, a brain, a liver, a heart, a lung, a tumor, or a combination thereof). The hollow organ may be a vascular section, e.g., an artery and/or vein. The first image may advantageously map an initial, in particular pre-procedural and/or static state of the object under examination, in particular of the anatomical structure.

The second image may map the spatial and/or time-related change in the object under examination in 2D and/or 3D spatial resolution. The second image may also map the spatial and/or time-related change in the object under examination in a time-resolved manner. Advantageously, the second image may have a plurality of second picture elements, in particular pixels and/or voxels. The second picture elements of the second image may each have a time intensity curve which maps the change in the object under examination as an intensity change over time. The change in the object under examination may be both a spatial and a time-related change with respect to the initial, in particular pre-procedural, state of the object under examination. The change in the object under examination may include a movement of a contrast agent and/or of a medical object. Advantageously, the second image may map an intraprocedural state of the object under examination, in particular of the anatomical structure.

The first and/or the second image may be a grayscale image in each case.

The display unit may include a screen, a monitor, a projector, a projection surface, or a combination thereof. The display surface of the display unit may include the projection surface and/or a display panel of the screen and/or of the monitor. In particular, the display surface of the display unit may include a spatially limited, in particular contiguous, surface on which the graphical representations may be displayed in chronological sequence.

In act c), a graphical representation of the first or second image is displayed on the display surface of the display unit. The graphical representation of the first or second image may be displayed with 2D and/or 3D spatial resolution on the display surface by the display unit. In addition, the graphical representation of the first or second image may be displayed in a time-resolved manner on the display surface by the display unit. In act d), a graphical representation of the respective other image, in particular the image whose graphical representation was not displayed in act c), is displayed on the display surface of the display unit. Acts c) and d) are carried out one after the other in terms of time. As a result, the graphical representations of the first and second images are displayed one after the other in terms of time, (i.e., not simultaneously), on the display surface of the display unit. Advantageously, the display unit may display the graphical representations of the first and second images in grayscale on the display surface.

In addition, acts b) to d) are carried out repeatedly. In particular, during the repeated execution of acts b) to d), a further second image is provided in each case, which maps the spatial and/or time-related change in the object under examination at a later point in time than the respective second image previously provided. In act c) or d), during the repeated execution of acts b) to d), a graphical representation of the second image provided last in each case may be displayed on the display surface of the display unit. In particular, the graphical representations of the first image and the in particular last provided second image may be displayed alternately on the display surface of the display unit.

Act b) is advantageously carried out before or after the display of the graphical representation of the first image, so that no further second image is acquired while the graphical representation of the first image is being displayed. This may advantageously minimize the radiation exposure of the object under examination.

The proposed embodiment may provide improved monitoring of spatial and/or time-related changes in the object under examination. By the repeated execution of acts b) to d), the states of the object under examination that are reproduced by the first and second images may be acquired and visually compared by the user. In particular, the repeated, in particular alternating, display of the graphical representations of the first and second images may result in an, in particular, lossless temporal mixing, in particular superimposition, of the graphical representations in the visual perception of the user, in particular due to a limited perception rate on the part of the user.

In a further advantageous embodiment of the proposed method, the graphical representation in act c) may be displayed with a first display duration and the graphical representation in act d) may be displayed with a second display duration. The first and second display durations may be the same or different.

The first display duration may describe a first time period, wherein during the first time period the graphical representation in act c) is displayed, in particular continuously, on the display surface of the display unit. The first display duration may characterize the length of the first time period. In addition, the second display duration may describe a second time period, wherein during the second period, the graphical representation in act d) is displayed, in particular continuously, on the display surface of the display unit. The second display duration may characterize the length of the second time period.

According to a first variant, the first and second display durations may be the same, in particular the first and second time periods may have the same length. In this case, the graphical representations of the first and the second image may be displayed alternately in acts c) and d), each with an identical, in particular first and second, display duration on the display surface of the display unit.

According to a second variant, the first and second display periods may be different, in particular the first and second periods may have different lengths. In this case, the graphical representations of the first and second images may be displayed alternately in acts c) and d), each with a different, in particular first and second, display duration, on the display surface of the display unit.

The first and second display durations may be regarded as weighting factors of the temporal mixing of the graphical representations of the first and second images. The first variant may be understood as equal weighting of the graphical representations of the first and second images in the temporal mixing. The second variant may be understood as a different weighting, in particular unequal weighting, of the graphical representations of the first and second images in the temporal mixing. In the second variant, for example, a proportion of the graphical representation of the first or second image having the comparatively longer display duration may be weighted more heavily in the temporal mixing.

In another advantageous embodiment of the proposed method, a repetition rate of the repeated execution of acts b) to d) and/or the first display duration and/or the second display duration may be adjusted such that the graphical representations displayed in acts c) and d) are not temporally separable for a human user, (e.g., at a perception rate of at most 25 images per second).

The repetition rate of the repeated execution of acts b) to d) may predefine a time frequency until the next execution of acts b) to d) or a maximum time duration for each execution of acts b) to d). If act b) is executed at least partially simultaneously with act c) and/or d), the repetition rate may predefine a sum of the first and second display durations.

The repetition rate of the repeated execution of acts b) to d) and/or the first display duration and/or the second display duration may characterize, in particular predefine, an image repetition rate of the alternating display of the graphical representations of the first and the second image on the display surface of the display unit. The image repetition rate of the alternate display of the graphical representation of the first and second images may be characterized by the number of repeated executions of acts c) and d) within a predetermined period of time, for example one second.

A human user may be trained to perceive an alternating display of graphical representations with a predetermined perception rate. The predetermined perception rate refers to a maximum number of individual images of the alternating display that may be perceived separately by the user. For most human users, the predetermined perception rate is limited to a maximum of 25 images per second.

Advantageously, the repetition rate of the repeated execution of acts b) to d) and/or the first display duration and/or the second display duration may be adjusted such that the graphical representation displayed in acts c) and d), in particular alternatingly, are not time-separable for the human user, in particular cannot be perceived separately. In other words, the image repetition rate and/or the first display duration and/or the second display duration may be adjusted such that the image repetition rate of the alternating display of the graphical representations of the first and the second image on the display surface of the display unit is more than 25 images per second.

The proposed embodiment may advantageously enable the user to temporally mix the graphical representations of the first and second images. The adjusted repetition rate and/or first display duration and/or second display duration may provide smooth and, advantageously, non-separable perception of the graphical representations of the first and second images by the user.

In a further advantageous embodiment of the proposed method, an image repetition rate of the display of the graphical representations in acts c) and d) may be at least 50 images per second.

With an image repetition rate of the, in particular alternating, display of the graphical representations of the first and second images in acts c) and d) of at least 50 images per second, the graphical representation of the first image and the graphical representation of the second image, in particular the second image provided last in each case, may each be displayed at least 25 times within one second on the display surface of the display unit.

This makes it possible to display the graphical representations of the first and second images particularly smoothly, in particular without jerkiness.

In a further advantageous embodiment of the proposed method, a first user input may be recorded. The first and/or the second display duration may be adjusted based on the first user input.

Advantageously, the first user input may be recorded by an input unit, (e.g., a keyboard and/or a switch such as a button and/or foot switch), and/or an input display, (e.g., a capacitive and/or resistive input display), and/or a microphone and/or a camera for gesture recognition. The first user input may specify a value for the first and/or second display duration. Alternatively, the first user input may include a setting for the adjustment, (e.g., relative and/or absolute adjustment), in particular a shortening or lengthening, of the first and/or second display duration. Advantageously, the first and/or the second display duration may be adjusted, in particular shortened or lengthened, based on the first user input.

The proposed embodiment may allow user-side adjustment of the weighting of the graphical representation of the first and second image for the temporal mixing thereof, because a ratio of the first and second display duration corresponds to a weighting ratio of the temporal mixing.

In a further advantageous embodiment of the proposed method, the change may include a movement of a medical object and/or of a contrast agent in the object under examination.

The change may include a movement of an, in particular, contrast-enhancing and/or intensity-enhancing contrast agent, for example, a spreading and/or flowing movement of the contrast agent, in particular a contrast agent flow and/or a movement of a contrast agent bolus, in the object under examination. Alternatively, or in addition, the change may include a movement of a medical object in the object under examination. The medical object may take the form of an endoscope, (e.g., a laparoscope), and/or a catheter and/or a guide wire and/or an implant, (e.g., a stent). At least part of the medical object, in particular a distal section of the medical object, may advantageously be disposed intraprocedurally in the object under examination. The movement of the medical object may include for example translation and/or rotation of the medical object, in particular of the distal section, in the object under examination.

Advantageously, the second image may map the medical object and/or the contrast agent as the spatial and/or time-related change in the object under examination. In the repeated execution of acts b) to d), the second images may map the movement of the medical object and/or the contrast agent as the spatial and/or time-related change in the object under examination.

The proposed embodiment may provide improved monitoring of contrasted anatomical structures of the object under examination and/or movement of a medical object in the object under examination.

In a further advantageous embodiment of the proposed method, the anatomical structure may be a hollow organ. In this case, the second image may map the change in the hollow organ.

The anatomical structure may advantageously include a hollow organ, vascular section, and/or vascular tree of the object under examination, for example, an artery and/or a vein. The second image may map the spatial and/or time-related change, in particular the movement of the medical object and/or of the contrast agent, in the hollow organ.

The proposed embodiment may provide improved navigation, (e.g., during the movement and/or positioning of a medical object), in the hollow organ of the object under examination.

In a further advantageous embodiment of the proposed method, the first and second images may be registered with each other, in particular repeatedly.

Advantageously, the second image provided last in each case may be registered with the first image. The registration of the first and the second image may be based on common anatomical and/or geometric features of the object under examination which are shown in the first and the second image. Alternatively, or in addition, the registration of the first and the second image may be based on acquisition parameters for acquiring the first and the second image, for example, an acquisition geometry of the medical imaging device with respect to the object under examination during acquisition of the first and the second image.

The registration of the first and second images may include determining and applying a transformation rule to the second image, in particular the second image provided last in each case. The transformation rule may predefine a translation, a rotation, a scaling, a deformation, or combination thereof of the second image. Determining the transformation rule may advantageously involve minimizing any deviation between the common anatomical and/or geometrical features of the object under examination shown in the first and the second image. Alternatively, or in addition, the determination of the transformation rule may be based on a correspondence between the acquisition geometry for acquiring the first and second image. Advantageously, the registration of the first and second images may include motion correction, in particular to correct a physiological movement of the object under examination.

As a result, the anatomical and/or geometric features of the object under examination shown in the first and second images may advantageously be displayed congruently in the alternating display of the graphical representations of the first and second images on the display surface of the display unit. This may also minimize artefacts, (e.g., motion artefacts), in the temporal mixing of the graphical representations of the first and second images.

In a further advantageous embodiment of the proposed method, a further user input may be entered. The further user input may specify how often act c) is executed during the repeated execution of acts b) to d).

The further user input may advantageously be recorded by the input unit, in particular analogously to the entering of the first user input. Advantageously, the further user input may set a frequency of execution of act c) during the repeated execution of acts b) to d). The frequency of execution of act c) specified by the further user input may advantageously be less than the frequency of execution of act d) during the repeated execution of acts b) to d). In particular, the graphical representation of the most recently provided second image may be displayed on the display surface of the display unit for each execution of acts b) to d), while the graphical representation of the first image is only displayed for some of the executions of acts b) to d).

The further user input may specify the frequency of execution of act c), for example, as a ratio to the frequency of execution of act d). In particular, the further user input may specify the frequency of execution of act c) by specifying how many repeated executions of acts b) and d) take place before act c) shall be performed. In addition, the further user input may enable act d) to be executed as and when required.

Adjusting the frequency of the repeated execution of act c) enables the effective display duration of the graphical representation displayed in act c) to be adjusted with respect to the display duration of the graphical representation displayed in act d). As a result, a weighting of the temporal mixing of the graphical representations of the first and second images may be adjusted by the user, in particular without adjusting the first and second display durations.

In another advantageous embodiment of the proposed method, the provision of the first image may include acquiring a first mask image and a first fill image. The first image may be generated as a difference image from the first mask image and the first fill image.

The first mask image may map the object under examination, (e.g., the anatomical structure), in a first temporal phase, (e.g., a first mask phase). In addition, the first fill image may map the object under examination, (e.g., the anatomical structure), in a second temporal phase, (e.g., a first fill phase). The first fill phase may be placed after the first mask phase in terms of time. During the first fill phase, a contrast agent may be disposed in the object under examination, in particular the anatomical structure. The first fill image may map the contrast agent disposed in the object under examination, in particular the anatomical structure. Advantageously, the first image may be generated from the first mask image and the first fill image as a difference image, for example, by subtracting the first mask image from the first fill image. For this purpose, the first mask image and the first fill image may advantageously be registered with each other, for example, on the basis of common anatomical and/or geometric features shown in the first mask image and the first fill image, and/or on the basis of an acquisition geometry for acquiring the first mask image and the first fill image.

As a result, the first image may advantageously have a contrasted mapping of an anatomical structure, (e.g., a hollow organ), of the object under examination, e.g., with uncontrasted areas not being shown in the first image.

In a further advantageous embodiment of the proposed method, the provision of the second image may include acquiring a second fill image. The second image may be generated as a difference image from the first mask image and the second fill image.

Advantageously, the second fill image may map the object under examination in a fourth temporal phase, in particular a second fill phase. The second fill phase may be placed after the first mask phase and the first fill phase in terms of time. During the second fill phase, a contrast agent and/or a medical object may be disposed in the object under examination, in particular a hollow organ of the object under examination. The second fill image may map the medical object and/or the contrast agent disposed in the object under examination. In addition, the second image may be generated from the first mask image and the second fill image as a difference image, for example, by subtracting the first mask image from the second fill image. For this purpose, the first mask image and the second fill image may advantageously be registered with each other, for example, on the basis of common anatomical and/or geometric features shown in the first mask image and the second fill image, and/or on the basis of an acquisition geometry for acquiring the first mask image and the second fill image.

By subtracting the first mask image from the second fill image, it is advantageously possible to dispense with acquiring a further mask image. This may minimize the duration of the examination and/or exposure, (e.g., radiation exposure), for the object under examination.

In a further advantageous embodiment of the proposed method, the provision of the second image may include acquiring a second mask image and a second fill image. The second image may be generated as a difference image from the second mask image and the second fill image.

The second mask image may map the object under examination in a third temporal phase, in particular a second mask phase. In addition, the second fill image may map the object under examination in a fourth temporal phase, in particular a second fill phase. The second fill phase may be placed after the second mask phase in terms of time. In addition, the second mask phase may be placed after the first mask phase and the first fill phase in terms of time. During the second fill phase, a contrast agent and/or a medical object may be disposed in the object under examination, in particular a hollow organ of the object under examination. The second fill image may map the medical object and/or the contrast agent disposed in the object under examination. The second image may also be generated as a difference image from the second mask image and the second fill image, for example, by subtracting the second mask image from the second fill image. For this purpose, the second mask image and the second fill image may advantageously be registered with each other, for example, on the basis of common anatomical and/or geometric features shown in the second mask image and the second fill image and/or on the basis of an acquisition geometry for acquiring the second mask image and the second fill image.

As a result, the second image may advantageously map the temporal and/or spatial change in the object under examination, (e.g., the hollow organ), wherein areas that are temporally and/or spatially unchanged may not be shown in the second image.

In a further advantageous embodiment of the proposed method, the first and the second image may be acquired using the same or different medical imaging devices, in particular different imaging modalities.

The at least one medical imaging device for acquiring the first and second images may be configured as a magnetic resonance imaging (MRI) installation, a computed tomography (CT) installation, a medical X-ray device, a positron emission tomography (PET) installation, a ultrasound device, or a combination thereof.

This provides improved, in particular dedicated, mapping of the anatomical structures to be mapped in the first and second images and/or of the spatial and/or time-related changes in the object under examination.

In a second aspect, the disclosure relates to a system including a medical imaging device and a display unit. The medical imaging device is configured to provide a first image mapping an anatomical structure of an object under examination. The medical imaging device is also configured to repeatedly provide a second image showing a spatial and/or time-related change in the object under examination. The display unit has a display surface. The display unit is configured to repeatedly map a graphical representation of the first image and a graphical representation of the most recently provided second image on the display surface in chronological order.

The advantages of the proposed system correspond to the advantages of the proposed method for alternately displaying graphical representations. The features, advantages, or alternative embodiments mentioned herein are also applicable to the other claimed subject matter and vice versa.

In a third aspect, the disclosure relates to a computer program product including a computer program that may be loaded directly into a memory of a provision unit and having program sections for executing all the acts of the method for alternately displaying graphical representations when the program sections are executed by the provision unit.

The disclosure may further relate to a computer-readable storage medium on which program sections that may be read and executed by a provision unit are stored in order to carry out all the acts of the method for alternately displaying graphical representations when the program sections are executed by the provision unit.

The advantage of a largely software-based implementation is that even previously used provision units may be upgraded by a software update in order to operate in the manner according to the disclosure. In addition to the computer program, such a computer program product may also include additional elements such as documentation and/or additional components, as well as hardware components, such as hardware keys (dongles, etc.) for using the software.

### BRIEF DESCRIPTION OF THE DRAWINGS

Exemplary embodiments of the disclosure are shown in the accompanying drawings and are described in more detail below. The same reference characters are used for identical features in the different figures, in which:

FIGS. 1 to 5 schematically illustrate various advantageous embodiments of a proposed method for alternately displaying graphical representations.

FIG. 6 schematically illustrates an example of a proposed system.

### DETAILED DESCRIPTION

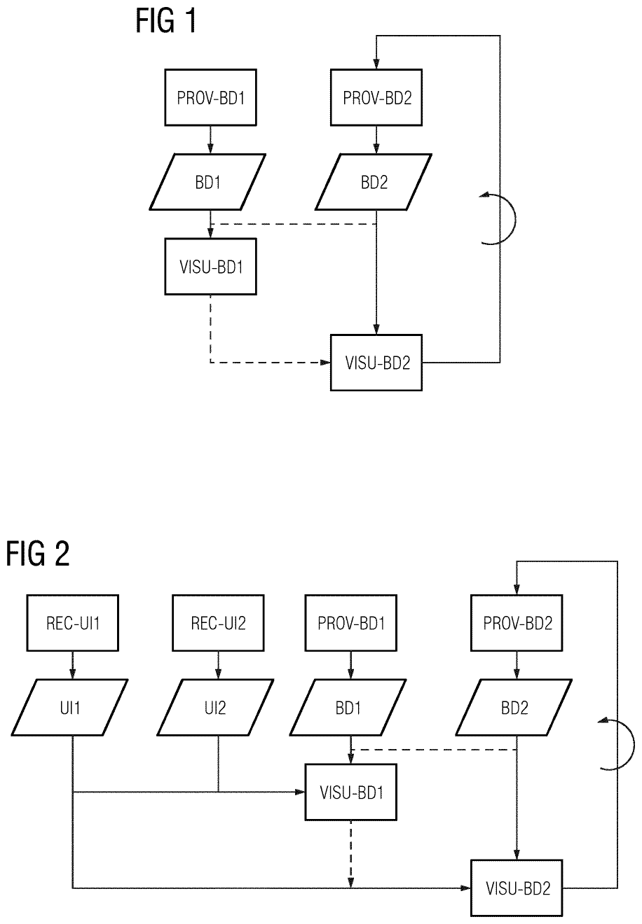

FIG. 1 schematically illustrates an advantageous embodiment of a proposed method for alternately displaying graphical representations. In act a), a first image BD1 may be provided PROV-BD1 that has a mapping of an anatomical structure of an object under examination. In act b), a second image BD2 may be provided PROV-BD2 that maps a spatial and/or time-related change in the object under examination. In a further act c), a graphical representation of the first image BD1 may be displayed on a display surface of a display unit VISU-BD1. In act d), a graphical representation of the second image BD2 may also be displayed on the display surface of the display unit VISU-BD2. Acts c) and d) may be carried out in chronological succession and acts b) to d) may be carried out repeatedly.

Advantageously, the graphical representation in act c) may be displayed with a first display duration and the graphical representation in act d) with a second display duration VISU-BD1, VISU-BD2. The first and second display durations may be the same or different.

In addition, a repetition rate of the repeated execution of acts b) to d) and/or the first display duration and/or the second display duration may be adjusted such that the graphical representations displayed in acts c) and d) are not temporally separable for a human user, e.g., having a perception rate of no more than 25 images per second. In particular, the image repetition rate of the display of the graphical representations in acts c) and d) may be at least 50 images per second.

The change in the object under examination may advantageously include a movement of a medical object and/or of a contrast agent in the object under examination. In addition, the anatomical structure may include a hollow organ, wherein the second image BD2 maps the change in the hollow organ.

Advantageously, the first image BD1 and the second image BD2 may be acquired using the same or different medical imaging devices, in particular imaging modalities.

FIG. 2 schematically illustrates another advantageous embodiment of a proposed method for alternately displaying graphical representations. A first user input UI1 may be recorded REC-UI1. In addition, the first and/or the second display duration may be adjusted based on the first user input UI1.

In addition, a further user input UI2 may be recorded REC-UI2. The further user input UI2 may specify how often act c) is executed during the repeated execution of acts b) to d).

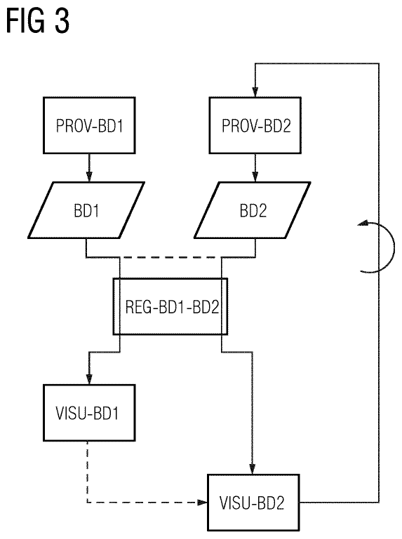

FIG. 3 schematically illustrates another advantageous embodiment of a proposed method for alternately displaying graphical representations. Here, the first image BD1 and the second image BD2 may be registered with each other, in particular repeatedly, REG-BD1-BD2.

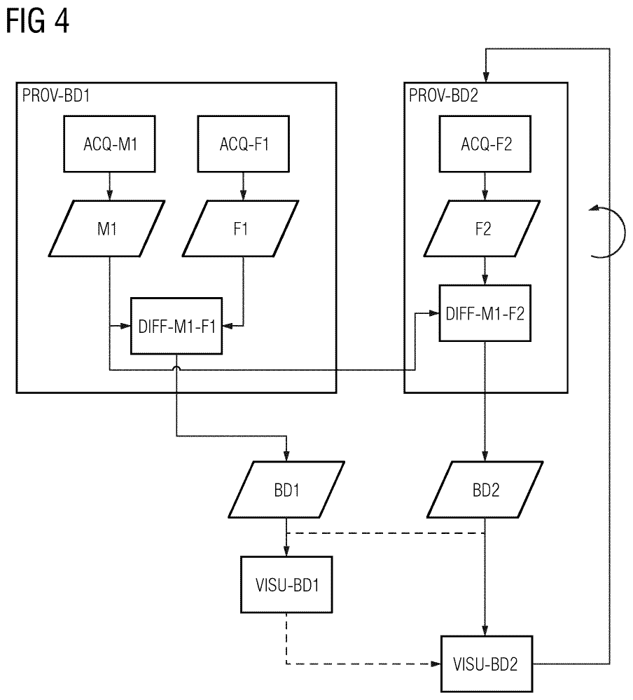

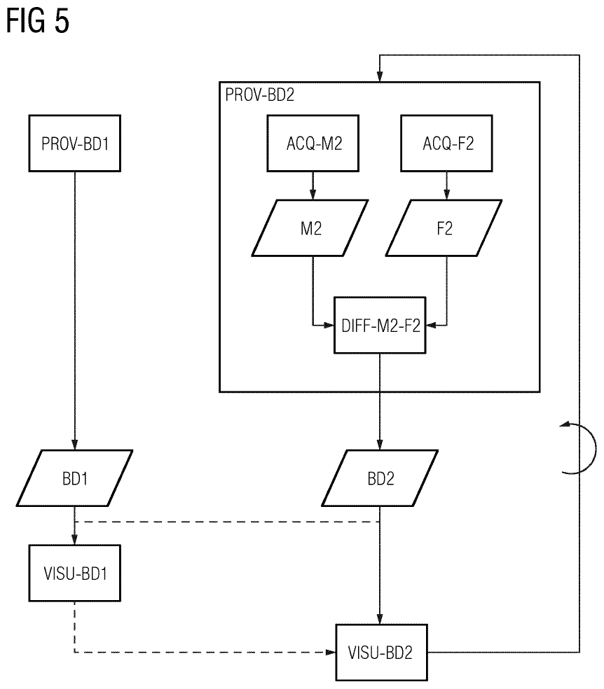

FIG. 4 schematically illustrates another advantageous embodiment of a proposed method for alternately displaying graphical representations. Here, the provision PROV-BD1 of the first image BD1 may include acquisition ACQ-M1 of a first mask image M1 and acquisition ACQ-F1 of a first fill image F1. The first image BD1 may be generated as a difference image DIFF-M1-F1 from the first mask image M1 and the first fill image F1.

The provision PROV-BD2 of the second image BD2 may include the acquisition ACQ-F2 of a second fill image F2. The second image BD2 may be generated from the first mask image M1 and the second fill image F2 as a difference image DIFF-M1-F2.

FIG. 5 schematically illustrates a further distributed embodiment of a proposed method for alternately displaying graphical representations. The provision PROV-BD2 of the second image BD2 may include the acquisition ACQ-M2 of a second mask image M2 and the acquisition ACQ-F2 of a second fill image F2. The second image BD2 may be generated from the second mask image M2 and the second fill image F2 as a difference image DIFF-M2-F2.

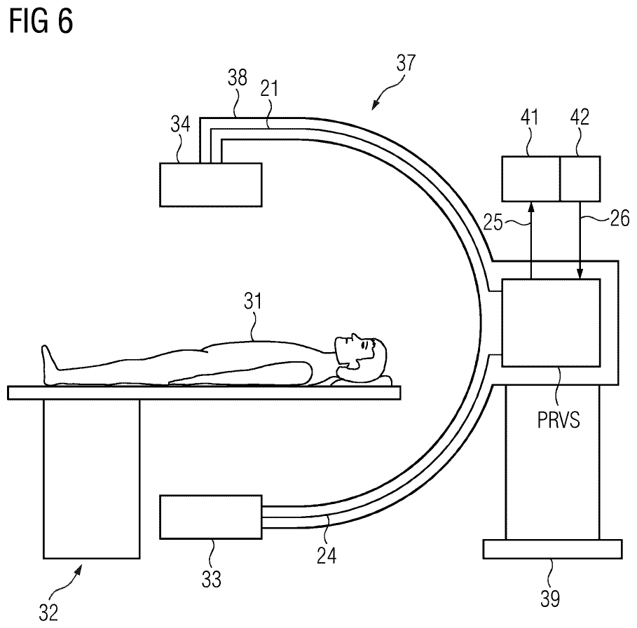

FIG. 6 schematically illustrates an advantageous embodiment of a proposed system. The system may include a medical imaging device and a display unit 41. In FIG. 6, a medical C-arm X-ray device 37 is shown schematically as an example of a medical imaging device. The medical imaging device, (e.g., the medical C-arm X-ray device 37), may be configured to provide PROV-BD1 the first image BD1. In addition, the medical imaging device, (e.g., the medical C-arm X-ray device 37), may be configured for repeated provision PROV-BD2 of the second image BD2.

The medical C-arm X-ray device 37 may advantageously include a detector 34, (e.g., an X-ray detector), and an X-ray source 33. To acquire the first image BD1 and the second image BD2, an arm 38 of the C-arm X-ray device 37 may be movably mounted about one or more axes. The medical C-arm X-ray device 37 may also include a movement apparatus 39 that enables the C-arm X-ray device 37 to move in space.

To acquire the first image BD1 and the second image BD2 of the object under examination 31 disposed on a patient positioning device 32, a provision unit PRVS may send a signal 24 to the X-ray source 33. The X-ray source 33 may then emit an X-ray beam. When the X-ray beam is incident upon a surface of the detector 34 after interaction with the object under examination 31, the detector 34 may send a signal 21 to the provision unit PRVS. On the basis of the signal 21, for example, the provision unit PRVS may receive the first image BD1 and/or the second image BD2.

In addition, the system may have an input unit 42, (e.g., a keyboard), and a display unit 41, (e.g., a monitor and/or a display and/or a projector). The input unit 42 may be incorporated in the display apparatus 41, for example, in the case of a capacitive and/or resistive input display. The input unit 42 may advantageously be configured to record the first user input REC-UI1 and/or the further user input REC-UI2. For this purpose, for example, the input unit 42 may transmit a signal 26 to the provision unit PRVS.

The display unit 41 may have a display surface. The display unit 41 may also be configured to repeatedly display, on the display surface, a graphical representation of the first image BD1 and a graphical representation of the last provided second image BD2 in chronological order VISU-BD1, VISU-BD2. For this purpose, the provision unit may transmit a signal 25 to the display unit 41.

The schematic representations contained in the figures described are not to scale or proportionally accurate.

Finally, it should be reiterated that the methods and apparatus described in detail above are merely exemplary embodiments which may be modified by a person skilled in the art in a wide variety of ways without departing from the scope of the disclosure. In addition, the use of the indefinite articles “a” or “an” does not exclude the possibility that the features in question may be present more than once. Likewise, the terms “unit” and “element” do not exclude the possibility that the components in question include a plurality of interacting subcomponents which may also be spatially distributed.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for alternately displaying graphical representations, the method comprising:
a) providing a first image that has a mapping of an anatomical structure of an object under examination;
b) providing a second image that maps a spatial change and/or time-related change in the object under examination;
c) displaying a graphical representation of the first image or second image on a display surface of a display unit; and
d) displaying a graphical representation of the respective other image on the display surface of the display unit,
wherein acts c) and d) are carried out one after the other in terms of time, and
wherein acts b) to d) are repeated.

2. The method of claim 1, wherein the graphical representation in act c) is displayed with a first display duration and the graphical representation in act d) is displayed with a second display duration.

3. The method of claim 2, wherein a repetition rate of the repeated execution of act b) to d) and/or the first display duration and/or the second display duration are adjusted such that the graphical representations displayed in acts c) and d) are not temporally separable for a human user.

4. The method of claim 3, wherein an image repetition rate of display of the graphical representations in acts c) and d) is at least 50 images per second.

5. The method of claim 2, wherein a first user input is recorded, and
wherein the first display duration and/or the second display duration is adjusted based on the first user input.

6. The method of claim 1, wherein the spatial change and/or the time-related change comprises a movement of a medical object and/or of a contrast agent in the object under examination.

7. The method of claim 6, wherein the anatomical structure comprises a hollow organ, and
wherein the second image maps the spatial change and/or the time-related change in the hollow organ.

8. The method of claim 1, wherein the first image and the second image are registered with each other.

9. The method of claim 1, further comprising:
recording a further user input,
wherein the further user input determines how often act c) is executed during the repeated execution of acts b) to d).

10. The method of claim 1, wherein the providing of the first image comprises acquisition of a first mask image and a first fill image, and
wherein the first image is generated from the first mask image and the first fill image as a difference image.

11. The method of claim 10, wherein the providing of the second image comprises acquisition of a second fill image, and
wherein the second image is generated from the first mask image and the second fill image as a difference image.

12. The method of claim 10, wherein the providing of the second image comprises acquisition of a second mask image and a second fill image, and
wherein the second image is generated from the second mask image and the second fill image as a difference image.

13. The method of claim 1, wherein the first image and the second image are acquired by a same medical imaging device.

14. A system comprising:
a medical imaging device; and
a display unit having a display surface,
wherein the medical imaging device is configured to:
provide a first image that maps an anatomical structure of an object under examination; and
repeatedly provide a second image mapping a spatial change and/or time-related change in the object under examination, and
wherein the display unit is configured to repeatedly display a graphical representation of the first image and a graphical representation of a most recently provided second image on the display surface in chronological order.

15. A computer program product comprising a computer program configured to be loaded directly into a memory of a provision unit, wherein the computer program, when executed by the provision unit, is configured to:
provide a first image that has a mapping of an anatomical structure of an object under examination;
repeatedly provide a second image that maps a spatial change and/or time-related change in the object under examination; and
repeatedly display a graphical representation of the first image and a graphical representation of a most recently provided second image on the display surface in chronological order.

16. The method of claim 3, wherein the human user has a perception rate of at most 25 images per second.

17. The method of claim 8, wherein the first image and the second image are repeatedly registered with each other.

18. The method of claim 1, wherein the first image and the second image are acquired by different medical imaging devices.
